(19) United States
(12) Patent Application Publication
(10) Pub. No.: US 2014/0187980 A1
(43) Pub. Date: Jul. 3, 2014

(54) Hypotube Sensor Mount for Sensored Guidewire

(71) Applicant: Volcano Corporation, San Diego, CA (US)

(72) Inventor: David H. Burkett, Temecula, CA (US)

(73) Assignee: Volcano Corporation, San Diego, CA (US)

(21) Appl. No.: 14/137,364

(22) Filed: Dec. 20, 2013

Related U.S. Application Data
(60) Provisional application No. 61/747,125, filed on Dec. 28, 2012.

(51) Int. Cl. A61B 5/0215

(52) U.S. Cl. CPC A61B 5/0215
USPC 600/486; 29/825

(57) ABSTRACT

A guidewire system for treating a patient may include a sensor assembly for detecting a physiological characteristic of a patient, the sensor assembly having a portion having a first width. The system also may include a hypotube having an integrated sensor mount formed therein for predictably locating the sensor during assembly, the hypotube having a lumen and the sensor mount being formed of opposing walls of the hypotube, the distance between the opposing walls being a second width. The first width of the sensor assembly may be greater than the second width between the opposing walls of the hypotube such that a portion of the sensor assembly lies directly on the walls of the hypotube. A sensor housing disposed about the sensor mount and configured to reinforce the hypotube at the sensor mount.

### CROSS-REFERENCE TO RELATED APPLICATIONS

The present application claims priority to and the benefit of U.S. Provisional Patent Application No. 61/747,125, filed Dec. 28, 2012, which is hereby incorporated by reference in its entirety.

### TECHNICAL FIELD

The present disclosure relates to intravascular devices, systems, and methods. In some aspects the present disclosure relates to intravascular devices, systems, and methods that include a hypotube having an integrated sensor mount.

### BACKGROUND

With the advent of angioplasty, pressure measurements have been taken in vessels and particularly in coronary arteries for the treatment of certain ailments or conditions. Typically in the past, such pressure measurements have been made by measuring the pressure at a proximal extremity of a lumen provided in a catheter advanced into the coronary artery of interest. Such an approach has, however, been less efficacious as the diameters of the catheters became smaller with the need to advance the catheter into smaller vessels and to the distal side of atherosclerotic lesions. This made necessary the use of smaller lumens that gave less accurate pressure measurements and in the smallest catheters necessitated the elimination of such a pressure lumen entirely. Furthermore, the catheter is large enough to significantly interfere with the blood flow and damp the pressure resulting in an inaccurate pressure measurement. In an attempt to overcome these difficulties, ultra miniature pressure sensors have been proposed for use on the distal extremities of a guidewire. Using a guidewire with a smaller diameter is less disruptive to the blood flow and thus provides an accurate pressure reading.

However the manufacturing process to consistently locate miniature sensors in guidewires can be challenging. For example, because of their size, current sensors on guidewires are mounted by hand in a housing cutout or mounted along a core wire. However, the optimal alignment of the sensor is dependent upon an assembler's ability to align the sensor within a given design. Because the sensors are often placed by hand, there is frequently some variability in sensor location from guidewire to guidewire. This variability may be compounded when sensors are located or placed by different workers.

Accordingly, there remains a need for improved devices, systems, and methods that have a capacity for increased consistency among workers even when the systems, devices, and methods are performed by hand. The present disclosure addresses one or more of the problems in the prior art.

### SUMMARY

In an exemplary aspect, the present disclosure is directed to a guidewire system for treating a patient. The system may include a sensor assembly for detecting a physiological characteristic of a patient, and may include a hypotube having an integrated sensor mount formed therein for predictably locating the sensor during assembly. The sensor mount may have a first mechanical stop configured to limit movement of the sensor in at least a first dimension and a second mechanical stop configured to limit movement of the sensor in at least a second dimension. A sensor housing may be disposed about the sensor mount and configured to reinforce the hypotube at the sensor mount.

In an aspect, the sensor assembly has a width greater than a width of a lumen of the hypotube and the sensor mount comprises walls of the hypotube such that a portion of the sensor assembly lies directly on the walls of the hypotube. In an aspect, the first mechanical stop is configured to maintain the sensor at a desired height, and wherein the second mechanical stop is configured to maintain the sensor at a desired axial location. In an aspect, the sensor housing comprises a window configured to provide fluid communication between the sensor and an environment outside the sensor housing. In an aspect, the integrated sensor mount comprises a cutout having a first level and a second level, the sensor assembly being disposed on the first level, the second level being lower than the first level. In an aspect, the sensor assembly extends longitudinally from the first level to a cantilevered position over the second level. In an aspect, the integrated sensor mount comprises a third level, the first and third level forming the first mechanical stop. In an aspect, the second mechanical stop is formed of upwardly facing surfaces of walls of the hypotube. In an aspect, the hypotube is formed of Nitinol and the sensor housing is formed of stainless steel. In an aspect, the system includes a flexible member disposed in the sensor mount, the flexible member comprising a more flexible distal end a less flexible proximal end, the flexible member extending from a distal end of the hypotube. In an aspect, the flexible member comprises an anchoring element disposed at a proximal end, the anchor member preventing the proximal end from passing out of the distal end of the hypotube.

In another exemplary aspect, the present disclosure is directed to a guidewire system for treating a patient. The system may include a sensor assembly for detecting a physiological characteristic of a patient, the sensor assembly having a portion having a first width. The system also may include a hypotube having an integrated sensor mount formed therein for predictably locating the sensor during assembly, the hypotube having a lumen and the sensor mount being formed of opposing walls of the hypotube, the distance between the opposing walls being a second width. The first width of the sensor assembly may be greater than the second width between the opposing walls of the hypotube such that a portion of the sensor assembly lies directly on the walls of the hypotube. A sensor housing disposed about the sensor mount and configured to reinforce the hypotube at the sensor mount.

In an aspect, the sensor mount comprises a first mechanical stop configured to limit movement of the sensor in at least a first dimension and a second mechanical stop configured to limit movement of the sensor in at least a second dimension. In an aspect, the sensor housing comprises a window configured to provide fluid communication between the sensor and an environment outside the sensor housing. In an aspect, the integrated sensor mount comprises a cutout having a first level and a second level, the sensor assembly lying on walls of the hypotube forming the first level, the second level being lower than the first level. In an aspect, the sensor assembly extends longitudinally from the first level to a cantilevered position over the second level. In an aspect, the integrated sensor mount comprises a third level, the first and third level forming a mechanical stop.

In another exemplary aspect, the present disclosure is directed to a method of building a guidewire. The method may include providing a sensor mount in a hypotube sized for introduction to a patient's vasculature when treating a medical condition; placing a sensor assembly on the sensor mount in the hypotube, the sensor mount having a surface configured to cooperate with the sensor assembly to locate the sensor assembly at a desired height by limiting movement of the sensor assembly in a first direction; orienting the sensor assembly to abut a mechanical stop that limits movement of the sensor assembly in a second dimension; securing the sensor in place; and introducing the sensor mount into a sensor housing having a window formed therein to increase the rigidity of the hypotube at the sensor mount.

In an aspect, the method may include aligning the sensor assembly with edges of the sensor mount to orient the sensor assembly in a third dimension. In an aspect, the method may include introducing a flex wire through an end of the hypotube to provide a flexible distal tip of the guidewire.

In another exemplary aspect, the present disclosure is directed to a method of building a guidewire. The method may include providing a sensor mount in a hypotube sized for introduction to a patient's vasculature when treating a medical condition; placing a sensor assembly on the sensor mount in the hypotube, the sensor assembly having a portion having a first width and spanning a lumen in the hypotube such that a portion of the sensor assembly lies directly on opposing walls of the hypotube; and securing the sensor in place; and introducing the sensor mount into a sensor housing having a window formed therein to increase the rigidity of the hypotube at the sensor mount.

In an aspect, the method may include aligning the sensor assembly with edges of the sensor mount to orient the sensor assembly in a third dimension. In an aspect, the integrated sensor mount comprises a cutout having a first level and a second level, and wherein introducing the sensor assembly includes placing the sensor assembly so that it extends longitudinally from the first level to a cantilevered position over the second level.

In another exemplary aspect, the present disclosure is directed to a guidewire system for treating a patient. The system may include a pressure sensor for detecting a physiological characteristic of a patient, and may include a hypotube having an integrated sensor mount formed therein for predictably locating the pressure sensor during assembly. The sensor mount may be disposed between two fully cylindrical portions of the hypotube. The sensor mount may have a first mechanical stop configured to limit movement of the sensor in at least a first dimension and a second mechanical stop configured to limit movement of the sensor in at least a second dimension, wherein the first mechanical stop is configured to maintain the sensor at a desired height, and wherein the second mechanical stop is configured to maintain the sensor at a desired axial location. The sensor may have an axial length greater than an axial length of the first mechanical stop so that the sensor extends as a cantilever from the first mechanical stop. A sensor housing may be disposed about the sensor mount and configured to reinforce the hypotube at the sensor mount. The sensor housing may comprise a window configured to provide fluid communication between the sensor and an environment outside the sensor housing. Conductors may extend inside the hypotube from the sensor to the proximal end of the hypotube. A stiffening portion may distally extend from one of the two fully cylindrical portions of the hypotube.

In an aspect, the first mechanical stop is an upper surface of walls of the hypotube.

### BRIEF DESCRIPTION OF THE DRAWINGS

Illustrative embodiments of the present disclosure will be described with reference to the accompanying drawings, of which:

FIG. 1 is a diagrammatic side view of a guidewire system according to an exemplary embodiment of the present disclosure.

FIG. 2 is a diagrammatic perspective view of a guidewire according to an exemplary embodiment of the present disclosure.

FIG. 3 illustrates a side view of a distal region of the guidewire of FIG. 2 according to an exemplary aspect of the present disclosure.

FIG. 4 illustrates a cross-sectional view of the a distal region of the guidewire of FIG. 2 according to an exemplary aspect of the present disclosure.

FIG. 5 illustrates an isometric view of a hypotube according to an exemplary aspect of the present disclosure.

FIG. 6 illustrates a cross-sectional view of a hypotube and sensor assembly according to an exemplary aspect of the present disclosure.

FIG. 7 illustrates a cross-sectional end view of a guidewire according to an exemplary aspect of the present disclosure.

FIG. 8 illustrates an isometric view of portions of a guidewire according to an exemplary aspect of the present disclosure.

FIG. 9 illustrates an isometric view of a flex wire according to an exemplary aspect of the present disclosure.

FIG. 10 illustrates an isometric view of a flex wire according to an exemplary aspect of the present disclosure.

FIG. 11 is a diagrammatic perspective view of a guidewire according to another embodiment of the present disclosure.

FIG. 12 illustrates an isometric view of a hypotube according to an exemplary aspect of the present disclosure.

FIG. 13 illustrates a side view of an integrated sensor mount of the hypotube of FIG. 12 according to an exemplary aspect of the present disclosure.

FIG. 14 illustrates a side view of an integrated sensor mount of the hypotube of FIG. 12 with a sensor assembly according to an exemplary aspect of the present disclosure.

FIG. 15 illustrates a cross-sectional end view of a guidewire according to an exemplary aspect of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any connections and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The devices, systems, and methods disclosed herein include a guidewire with an integrated sensor mount that is configured to increase the repeatability and consistency of sensor placement during the manufacturing process. In some embodiments, the sensor mount is arranged to enable a worker to locate the sensor at a precise height relative to the outer surfaces of the guidewire. In some embodiments, the sensor mount is arranged to enable a worker to locate the sensor at a precise distance in the axial direction from the distal end of the guidewire. In some embodiments, the sensor mount is arranged to enable a worker to reference the sensor mount when placing the sensor to identify the lateral position to increase consistency of assembly from guidewire to guidewire even among different workers. Some sensor mount embodiments allow a worker to locate the sensor in height, axial position, and lateral position. Accordingly, guidewires may be assembled with increased reliability and consistency. The guidewire having sensing capabilities may be adapted to be used in connection with a patient lying on a table or a bed in a cath lab of a typical hospital in which a catheterization procedure such as for diagnosis or treatment is being performed on the patient.

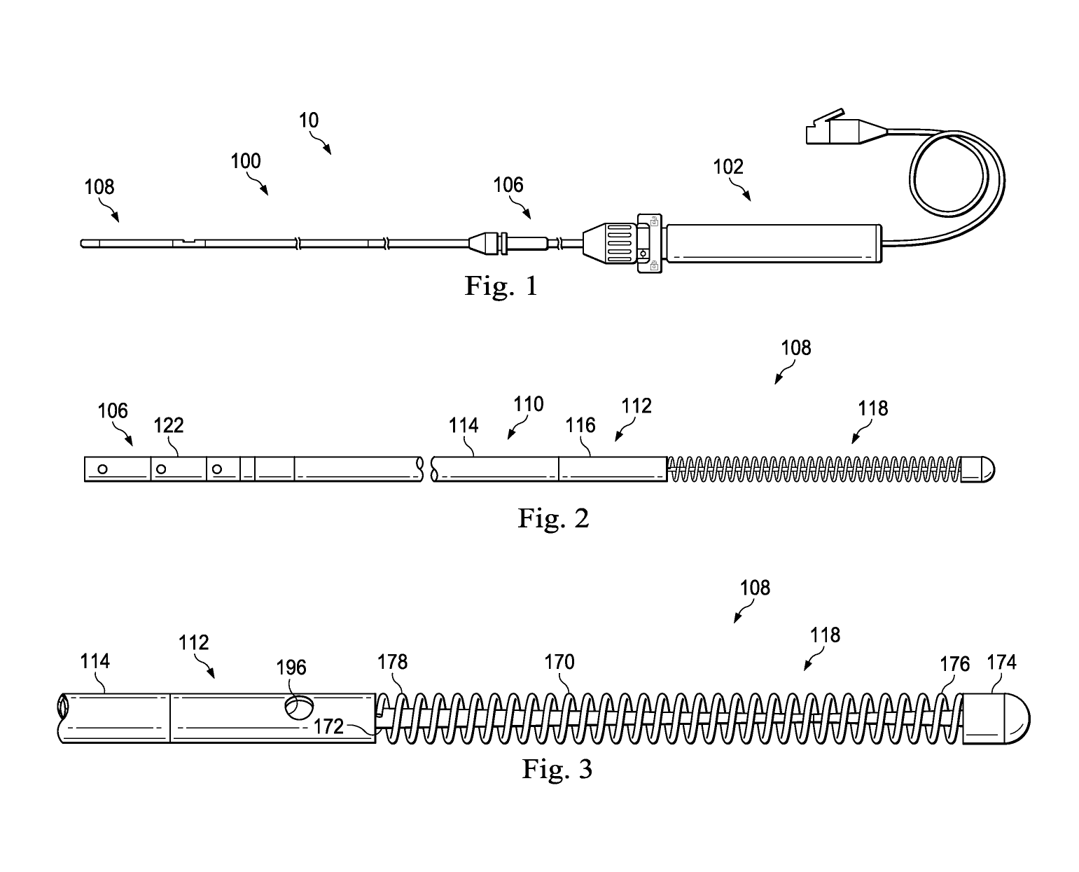

FIG. 1 shows an exemplary guidewire system 10 consistent with the principles disclosed herein. The guidewire system 10 in this embodiment is configured to sense or detect a physiological characteristic of a condition of the patent. For example, it may detect or sense a characteristic of the vasculature through which it has been introduced. In one embodiment, the guidewire system 10 has pressure sensing capabilities. The guidewire system 10 includes a guidewire 100 and a connector 102 disposed at the end of the guidewire 100. The connector 102 in this example in FIG. 1 is configured to communicate with the guidewire 100, serve as a grippable handle to enable the surgeon to easily manipulate the proximal end of the guidewire 100, and connect to a console or further system (not shown) with a modular plug. Accordingly, since the guidewire 100 is configured to detect physiological environmental characteristics, such as pressure in an artery for example, data or signals representing the detected characteristics may be communicated from the guidewire 100, through the connector 102, to a console or other system for processing. In this embodiment, the connector 102 is configured to selectively connect to and disconnect from the guidewire 100. In some embodiments, the guidewire system 10 is a single-use device The guidewire 100, in the embodiment shown, is selectively attachable to the connector 102 and includes a proximal portion 106 connectable to the connector 102 and a distal portion 108 configured to be introduced to a patient during a surgical procedure.

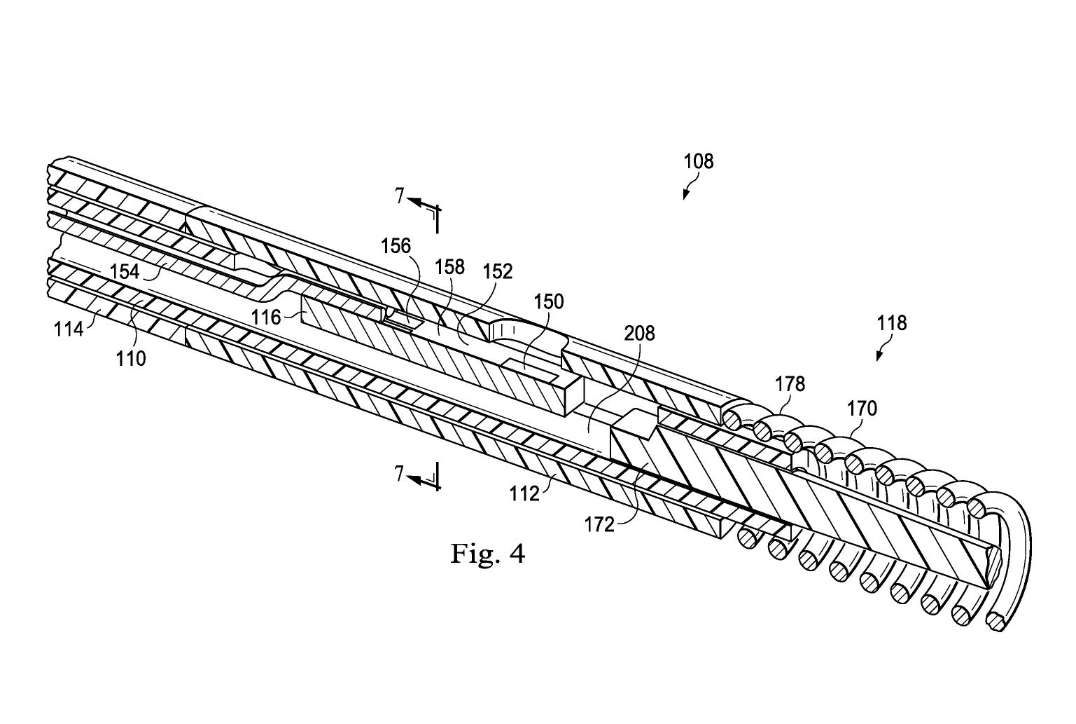

The guidewire 100 is shown in greater detail in FIGS. 2-4. FIG. 2 shows the entire guidewire 100, FIG. 3 shows the distal portion 108 of the guidewire 100, and FIG. 4 shows a cross-section of the distal portion 108 of the guidewire 100. Referring to these Figures, the guidewire includes a hypotube 110, a sensor housing 112, a proximal polymer sleeve 114, a sensor assembly 116, a distal tip 118, and a proximal electrical interface 122.

The proximal electrical interface 122 in FIG. 2 is configured to electrically connect the sensor assembly 116 and the connector 102 to order to ultimately communicate signals to the processing system. In accordance with this, the electrical interface 122 is in electrical communication with the sensor assembly 116 and in this embodiment is configured to be received within the connector 102. The electrical interface may include a series of conductive contacts on its outer surface that engage and communicate with corresponding contacts on the connector 102.

The sensor assembly 116 includes a sensor 150, a sensor block 152, and conductors 154 that extend from the sensor block 152 to the proximal electrical interface 122. The sensor 150 is arranged and configured to measure a physiological characteristic of a patient. When used on the guidewire 100, the sensor 150 is arranged and configured to measure a physiological characteristic of a vessel itself, such as a vascular vessel. In one embodiment, the sensor 150 is a pressure transducer configured to detect a pressure within a portion of a patient, such as the pressure within a blood vessel. In another embodiment, the sensor 150 is a flow control sensor that may be used to measure flow through the vessel. In yet other embodiments, the sensor 150 is a plurality of sensors arranged to detect a characteristic of the patient and provide feedback or information relating to the detected physiological characteristic. The sensor 150 may be disposed, for example, less than about 5 cm from the distal-most end of the guidewire 100. In one embodiment, the sensor is disposed about 3 cm from the distal-most end of the guidewire 100.

The sensor block 152 carries the sensor 150 and may be, for example, a wafer, a chip, or other transducer carrying substrate. The sensor block 152 in this embodiment is configured to carry the sensor 150 and configured to have contacts or conductive connectors 156 for communication with the conductors 154. The sensor block 152 in this embodiment is sized to fit within the diametric profile of the guidewire 100. In the embodiment shown, the sensor block 152 is relatively rectangular shaped and includes an outwardly facing sensor side 158 and an interface side 160 (FIG. 6) that is configured to engage and directly lie against the sensor mount 134. In this condition, the sensor block 152 may be particularly positioned in order to provide a consistent and predictable structure, reducing the chance of variation that may otherwise occur during manufacturing from employee to employee as the sensor block 152 is applied to the hypotube 110. The sensor block 152 may be sized to have an axial length in the range of about 0.020 to 0.055 inch. In one embodiment, the axial length is about 0.035. The width may be in the range of about 0.004 to 0.015 inch. In one embodiment, the width is about 0.009. The height may be in the range of about 0.001 to 0.008 inch. In one embodiment, the height is about 0.003 inch. Other sizes of sensor blocks are contemplated. The contacts 156 on the sensor block 152 may be formed at the proximal end and may be shaped to communicate electrically with the conductors 154. In the embodiment shown, the contacts 156 are disposed along the top surface of sensor side 158 of the sensor block 152, on the same side as the sensor 150. In alternative embodiments, the contacts 156 are disposed on a side opposite the sensor 150 on the interface side 160.

The connectors 154 extend from the contacts 156 to the proximal electrical interface 122 (FIG. 2). The connectors 154 are, in this embodiment, electrical cables or wires extending from the top of the sensor block 152. Since the contacts 156 are disposed on the same side of the sensor block 152 as the sensor 150, the connectors 154 extend from the top of the sensor block 152 rearward to the edge of the sensor block 152, and then bend to extend and enter the inner lumen of the hypotube 110. Since the guidewire 100 disclosed herein uses a hypotube, the system lacks a core and the connectors can extend in the hypotube lumen. The example shown employs three connectors 154, however the number of connectors in any particular embodiment may depend in part on the type or number of sensors disposed within the guidewire 100. In some embodiments, the connectors 154 are soldered to the contacts 156 on the sensor block 152 during the manufacturing process. Accordingly, the connectors 154 may carry signals to and from the sensor 150.

The hypotube 110 is a flexible elongate element having a proximal end region 130 and a distal end region 132 which are formed of a suitable biocompatible material. The proximal end region 130 extends to the proximal electrical interface 122. In some embodiments, the hypotube 110 is formed of a Nitinol alloy, while in other embodiments, the hypotube is formed of stainless steel. Other materials would be apparent to one of ordinary skill in the art. In some embodiments, the hypotube 110 has an outside diameter for example of 0.018 inch or less and has a suitable wall thickness of, for example, 0.002 inch to 0.005 inch, for example. Where a smaller guidewire is desired, the hypotube 110 can have an exterior diameter of 0.014 inch or less. Some embodiments of the guidewire system 10 use large-diameter hypotubes having an outer diameter in the range of about, for example, 0.025 inch to 0.040 inch. As such, the hypotube 110 may have a diameter in the range of about 0.040 inch or less. In large-diameter hypotubes, the inner diameter may be sized to be about half of the outer diameter. For example, a 0.035 inch outer diameter may have an inner diameter of about 0.016 inch. Likewise, an 0.018 inch outer diameter may have an inner diameter of about 0.010 inch. An 0.014 inch outer diameter may have a 0.007 inch inner diameter. Yet other sizes are also contemplated. In the embodiment shown, the smaller outer diameter may help the hypotube act as an alignment feature that enables a worker to properly locate the sensor assembly with reference to the hypotube. In some embodiments, the hypotube has a length of about 150-200 centimeters, although other lengths are contemplated.

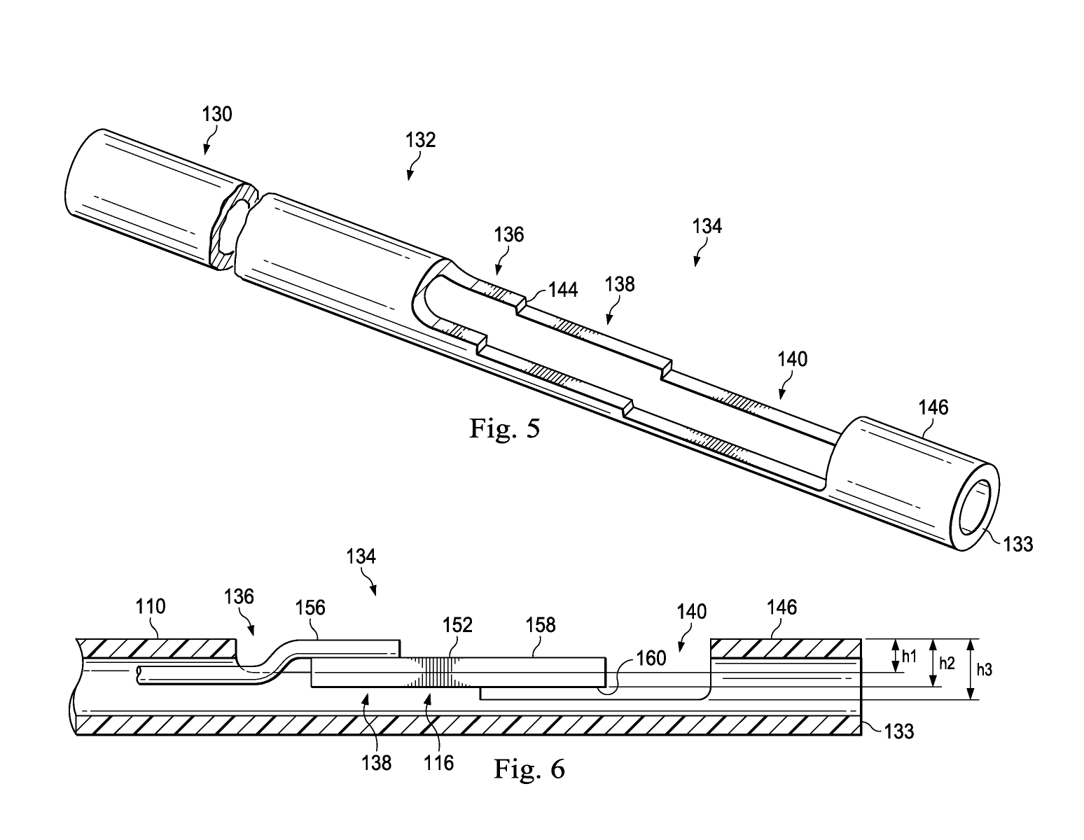

FIG. 5 shows the distal end region 132 of the hypotube 110. FIG. 6 shows the distal end region with the sensor assembly 116. In this embodiment, as shown in FIG. 5, the hypotube 110 includes a distal end 133 and includes an integrated sensor mount 134 formed therein. Here the sensor mount 134 is a cut-out formed within a side of the hypotube 110 to receive at least a part of the sensor assembly 116. The sensor mount 134 is particularly sized and configured to help accurately align the sensor 150 of the assembly 116 in the cutout. As discussed below, the geometry and size of the cutout as the sensor mount 134 can be used to precisely locate the sensor 150 vertically (or in a first dimension) and, in some embodiments, axially (or in a second dimension), while the walls of the cut hypotube 110 provide a visual reference for aligning the sensor 150 laterally (or in a third dimension). In addition, the hypotube diameter is designed to allow for a simpler external housing. Accordingly, the hypotube has an integral, built-in mounting feature.

The sensor mount 134 may be disposed about less than an inch from the distal end of the cylindrical portion of the hypotube 133. In this embodiment, the sensor mount 134 comprises a first region 136 having a first height h1, a second region 138 having a second height h2, and a third region 140 having a third height h3. The heights are shown in FIG. 6. The first region 136 in this case forms the proximal end of the sensor mount 134 and is disposed adjacent a completely enclosed or a completely cylindrical portion of the hypotube 110. The first region 136 has a height in the range of about 0.002″ to 0.005″, and may permit the first region 136 to accommodate the transmission carriers or conductors 154 that extend from the sensor block 152 to the proximal electrical interface 122. In some embodiments, the first region 136 starts at about 0.0630 inch from the distal end 133 and ends about 0.0710 inch from the distal end 133. However, other sizes and locations are contemplated.

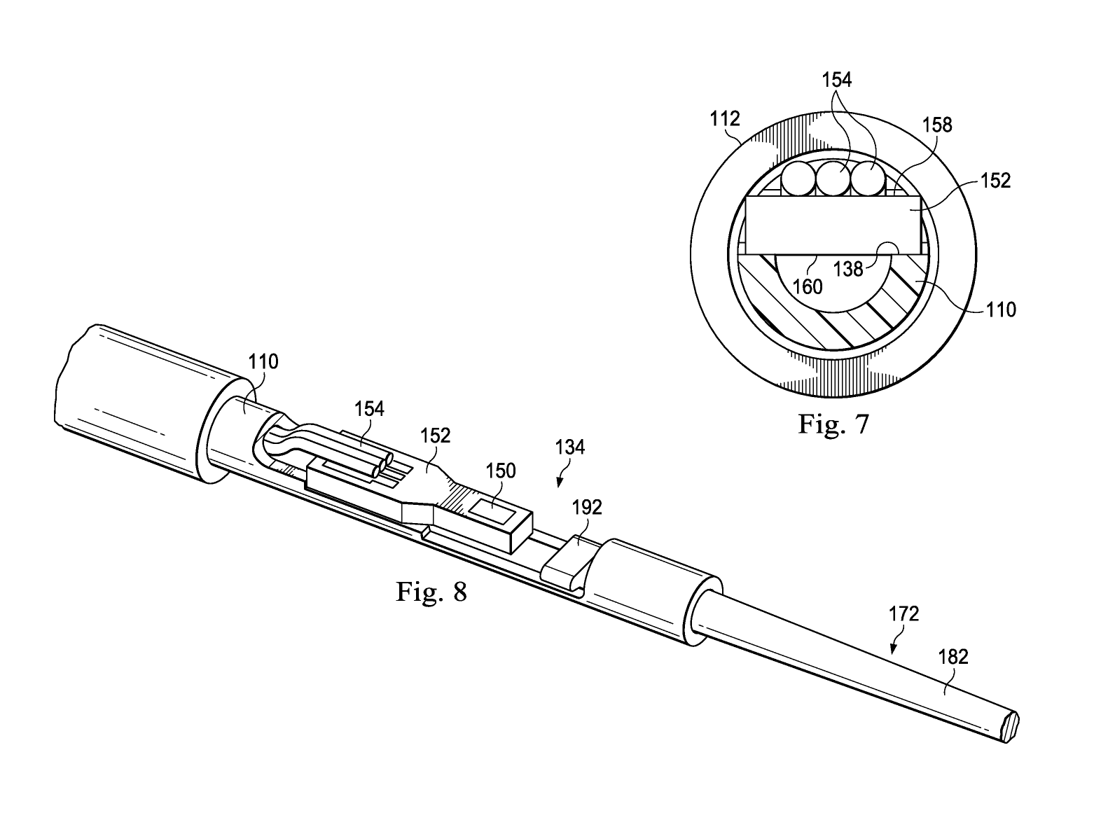

The second region 138 is arranged to simplify the assembly of the guidewire 100 by guiding the placement of the sensor block 152 onto the hypotube 110. The second region 138 is disposed distal of the first region 136 and proximal of the third region 140. The second region 138 is formed to actually receive or carry the sensor block 152. The height h2 of the second region 138 may be selected to precisely orient the sensor block 152 at the optimum height. For example, the height of the second region may be within the range of about 0.0030″ to 0.0060″ and may be selected based on the height of the sensor block 152. Other sizes are contemplated. In this embodiment, the diameter of the hypotube 110, and therefore, the width of the sensor mount 134 in the second region 138, is selected to correspond roughly with the width of the sensor block 152 so that the sensor block 152 can lie directly on the second region 138. FIG. 7 shows a transverse cross-sectional view taken through the second region 138 along lines 7-7 in FIG. 4. As can be seen in the cross-sectional view of FIG. 7, the sensor block 152 lies directly on the sidewalls of the hypotube forming the second region 138, and the second region 138 has a width just greater than the width of the sensor block 152. This enables a worker to easily align the sensor block 152 laterally relative to the second region 138 to substantially center the sensor block 152 on the second region of the hypotube 110. That is, the second region 138 is used as a reference to manually locate the sensor block 152 in a desired location, such as centered on the second region of the hypotube 110. As such, manufacturing efficiencies are achieved because the hand-assembled sensors may be placed directly against the second region 138 of the sensor mount 134. In addition, the second height is selected so that the sensor 150 sits at the optimum height, increasing reliability and reproducibility.

In one embodiment, the second region 138 starts at about 0.0460 inch from the distal end 133 and ends at about 0.0630 inch from the distal end 133 of the hypotube 110. As can be seen in FIG. 5, a distinct step 144 separates the first and second regions 136, 138. The step 144 may be used as a mechanical stop or mechanical reference during manufacturing in order to place the sensor block 152 in a desired location. Accordingly, in addition to having a particular height that holds the sensor block 152 at a particularly desired height, the step 144 separating the first and second regions 136, 138 may be used as a physical or mechanical stop against which the sensor block 152 may be set.

The third region 140 forms the distal end of the sensor mount 134 and is disposed adjacent a completely enclosed or a completely cylindrical portion 146 of the hypotube 110. The third region 140 has a height greater than the height of the second region 138. The height is greater than that of the second region 138 so that the sensor block 152 is cantilevered within the sensor mount 134. It's worth noting that although the height is greater in the third region 140, the third region is lower than the second region since height is measured as the depth of the cut into the hypotube 110. A cantilevered sensor block 152 may better isolate the sensor 150 from interference that may occur as a result of flexing of the hypotube that may occur as the guidewire 100 is fed through a patient's vasculature. That is, while the hypotube may flex, even along the sensor mount 134, the sensor readings may remain virtually unaffected because the sensor is cantilevered and therefore not subject to loading that may otherwise occur as a result of flexing of the hypotube 110. In some embodiments, the third region serves the dual purpose of also accommodating a stiffener that extends distally from the hypotube 110 as will be explained further below. In one embodiment, the third region starts about 0.0150 inch from the distal end 133 of the hypotube 110 and ends about 0.0460 inch from the distal end 133 of the hypotube 110. The distal cylindrical portion of the hypotube extends from the distal end 133 to about 0.0150″ from the distal end 133. Other dimensions are contemplated.

The proximal polymer sleeve 114 is disposed about the hypotube 110 and extends proximally from the sensor mount 134 toward the proximal electrical interface 122. In the exemplary embodiment shown, the polymer sleeve 114 is formed of a biocompatible polymeric material, such as Pebax®, for example, in order to reduce friction incurred as the guidewire is introduced through vessels in the body. Other materials may be used. Depending on the embodiment, the polymer sleeve 114 may have a thickness of about 0.001″ to 0.002″, although other thicknesses are contemplated. In the example shown, the sleeve may include a hydrophilic coating that also lubricates and enables low friction passage through the vessels.

The distal tip 118 includes a coil 170, a flex wire 172, and a distal cap 174. The coil 170 may be best seen in FIGS. 3 and 4 and extends from the distal end region 132 of the hypotube 110 in the distal direction to the distal cap 174. As such, the coil 170 includes a distal portion 176 and a proximal portion 178. The coil 170 may be a coil spring formed of a suitable material such as stainless steel or Nitinol, for example. In one embodiment, the coil 170 has an outside diameter of 0.018″ and is formed from a wire having a diameter of 0.003″. The proximal portion 178 is connected or attached, such as by threading, onto the distal end region 132 of the hypotube 110. The distal portion 176 of the coil 170 is secured about the distal cap 174. In some embodiments, the coil 170 is formed of a highly radiopaque material such as palladium or a tungsten platinum alloy. In some examples, it has a length within a range of about 20 cm to 30 cm, although other ranges are contemplated.

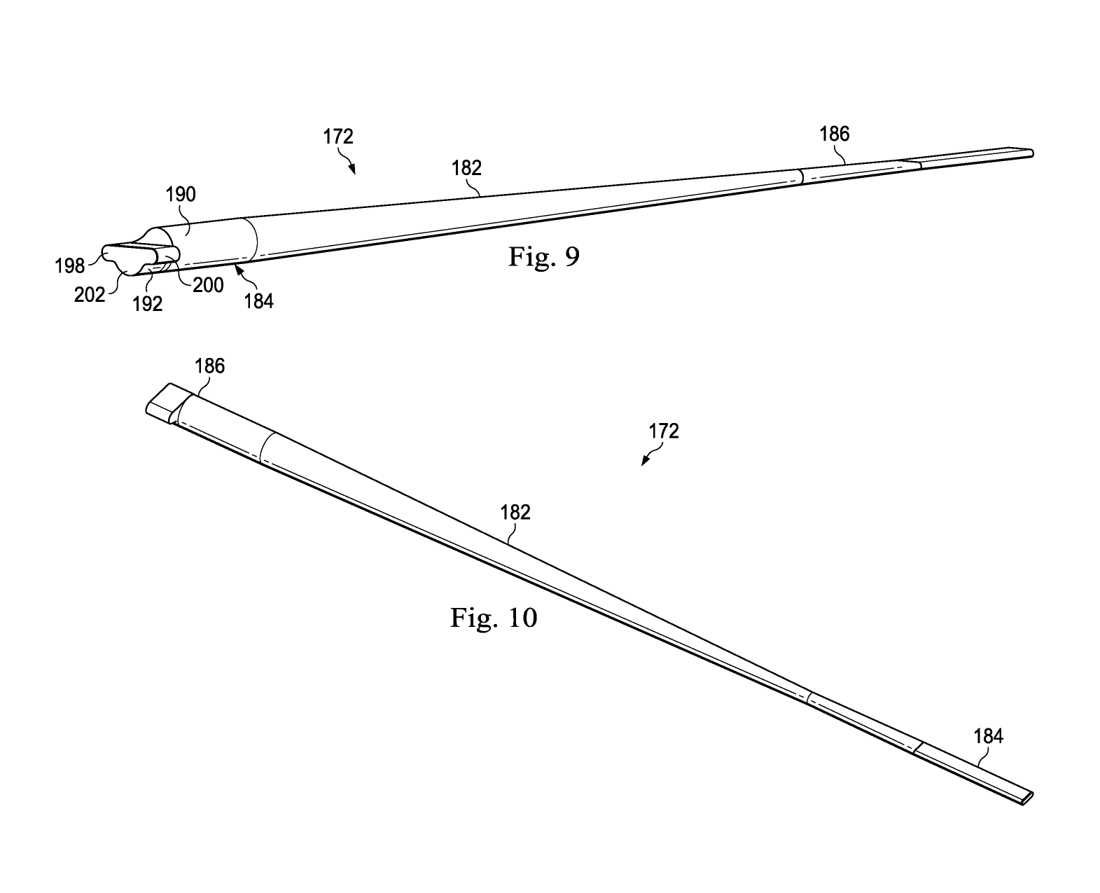

The flex wire 172 extends within an inner diameter of the coil 170 from the distal end region 132 of the hypotube 110. In the exemplary embodiment shown, the flex wire 172 cooperates with the sensor mount 134 to be secured in place. The flex wire 172 is shown in FIG. 8 attached to the hypotube 110 without the coil 170 and is shown in even greater detail in FIGS. 9 and 10. The flex wire 172 may be formed of any material suitable for bending while providing structural stability to the coil 170, including for example, a stainless steel wire, a Nitinol wire, or other biocompatible material.

The flex wire 172 is formed of a body 182 extending between and connecting a proximal end 184 and a distal end 186. The flex wire 172 flexes in order to traverse tortuous vessels in the patient's body. The body 182 tapers from the proximal end 184 to the distal end 186. Since the cross-section of the tapering body 182 decreases in the distal direction, the distal end has a greater flexibility than the proximal end. As such, the flex wire 172 may provide some stability and transition from more flexible in the distal direction to more stiff in the proximal direction. In the embodiment shown, the tapering body 182 is cylindrically shaped, thereby forming a conical taper. Other embodiments have other profiles. For example, some embodiments have a square cross-section, a rectangular cross-section, an oval cross-section, or other shape.

The proximal end 186 of the flex wire 172 has a region of constant diameter 190 and an anchoring element 192. The region of constant diameter 190 extends from the anchoring element 192 to the tapered body 182. The region of constant diameter 190 is sized and arranged to fit within the distal end region 132 of the hypotube 110. The anchoring element 190 is formed to have a width greater than the inner diameter of the distal end of the hypotube 110. Because of its size and profile, the anchoring element 192 has a width greater than the inner diameter of the end of the hypotube distal end region 132. Accordingly the anchoring element 192 is configured to abut against the proximal side of the distal end region 132 of the hypotube 132 and prevent the flex wire 172 from passing through and out of the distal end region 132 of the hypotube 110. In the embodiment shown, the anchoring element 192 comprises a first wing 198, a second wing 200, and a lug 202. At least the first and second wings 198, 200 extend wider than the diameter of the region of constant diameter 190 and wider than the inner diameter of the hypotube 110. They each include a flat side surface configured to abut against and rest upon the third region 140 of the sensor mount 134. In addition, the lug 202 is disposed to fit within the inner diameter of the hypotube 110. This is best seen in FIG. 7. The arrangement of the wings 198, 200 and the stabilizing lug 202 cooperate to prevent rotation of the flex wire 172 relative to the hypotube 110.

The distal cap 174 is disposed over the coil 170 and the flex wire 172 as shown in FIG. 3. In the example shown, the distal cap 174 has a leading rounded end that can smoothly slide against tissue as the guidewire 100 is fed through the vasculature of a patient. In this example, the distal cap 174 is a solder joint with a rounded end. In other embodiments, the distal cap 174 is a separate component secured to the coil 170 via an adhesive. However, in other embodiments, the distal cap 174 is secured to the coil 170 via welding or other attachment method.

The sensor housing 112 is disposed at the end of the polymer sleeve 114 and is configured to cover and protect the sensor assembly 116. As such, the sensor housing 112 covers the sensor mount 134 and forms a chamber 208 in which the sensor mount 134 resides. Since the stiffness of the hypotube 110 may be decreased by the sensor mount 134, the sensor housing 112 may be configured to restore the rigidity of the hypotube. In the embodiment shown, it does this by extending over and covering the cylindrical portions of the hypotube 110 at each end of the sensor mount 134, as can be seen in FIG. 4. The sensor housing 112 may be formed of a rigid material, such as a stainless steel, a nitinol alloy, or other biocompatible material that provides rigidity to the sensor mount region of the hypotube 110.

A window 196 in the sensor housing 112 provides fluid communication between the sensor assembly 116 in the chamber and the outer environment. In this embodiment, the window 196 is formed to lie directly above the sensor 150 is sized and configured so that the detected physiological characteristic at the sensor in the chamber 208 equates to the environmental characteristic outside the hypotube. For example, when the sensor 150 is a pressure sensor, the window 196 is sized so that the pressure in the chamber 208 about the pressure sensor 150 is substantially the same as the pressure outside the chamber 208.

Some embodiments of the sensor housing include a non-circular inner surface. Accordingly, the cross-section of the lumen may form an oval or other shape. In one embodiment, the oval shape accommodates sensor blocks that have a width greater than the outer profile of the hypotube with the sensor block is disposed on the sensor mount.

Assembly of the guidewire 100 may include obtaining the components or elements discussed above. In one embodiment, the integrated sensor mount 134 is formed in the hypotube 110 using a wire EDM cutting process, although other methods may be used. The worker may introduce the flex wire 172 into the sensor mount 134 so that the distal portion of the flex wire 172 and the body of the flex wire pass through and extend out of the distal end of the hypotube 110. As the flex wire 172 is advanced through the sensor mount 134 and the through the distal portion of the hypotube 110, the lug 202 on the flex wire 172 may be aligned to lie within the curved inner portion of the hypotube 110 and the wings 198, 200 may be disposed to lie upon upper surfaces of the third region 140 of the sensor mount 134 of the hypotube 110. The flex wire 172 may be advanced through the distal end of the hypotube 110 until the wings 198, 200 abut against the distal portion of the sensor mount 134. Accordingly, with the wings 198, 200 in place against the third region 140 of the sensor mount 134 and against the distal portion of the sensor mount 134, the flex wire 172 is positioned to be secured to the hypotube 110. The flex wire 172 may then be secured to the hypotube 110 by soldering. Other embodiments secure the flex wire 172 in place on the hypotube using an adhesive, welding, and other types of attachment methods.

With the flex wire 172 secured in the sensor mount 134, the sensor block 152 may be introduced to the sensor mount 134. The conductors 154 may be fed through the hypotube lumen to the sensor mount 134 to connect to the sensor block 152. The sensor block 152 carries the sensor 150 for detecting a physiological characteristic of a patient's vessel. As discussed above, in some embodiments, the sensor 150 is a pressure sensor. The sensor block 152 may have a width greater than an inner diameter of the hypotube 110 so that the sensor block 152 can lie directly on both sides of the sensor mount 134 in the manner shown in the cross-sectional view of FIG. 7. With the sensor block 152 lying on both sides of the sensor mount 134, the sensor block may be moved proximally until the distal end of the sensor block abuts against the step between the first region 136 and the second region 138 of the sensor mount 134. The second region 138 of the sensor mount 134 has a height that is selected to provide a height elevation to the sensor block 152 that is suitable for operation, and may be selected to place the sensor centrally in the chamber 208. Because the sensor 150 lies directly on the walls forming the hypotube sensor mount 134, variations in sensor height from guidewire to guidewire can be substantially reduced or eliminated. With the sensor height set by the sensor mount 134, and its axial location set by the abutment or step 133 between the first and region 136 and the second region 138, the worker can further align the sensor block 152 by visually comparing the lateral sides of the sensor block 152 to the lateral sides or edges of the hypotube 110. Accordingly, the sensor mount 134 provides a mechanical stop or mechanical limit to aid a worker in consistently placing the sensor at the same height and at the same axial position from guidewire to guidewire. In addition, the sensor mount provides a guide in the form of edges of the hypotube that enables the worker to visually align the sensor block 152 in the lateral direction. Accordingly, the worker may be able to produce product with greater precision and consistency than in prior designs.

The sensor block 152 may be secured in place using an adhesive or other securing method, such as those discussed above. With the sensor block 152 now secured in place, the conductors 154 may be connected to the contacts 156 on the sensor block 152. In some embodiments, these are soldered to the contacts 156, however other attachment methods are contemplated to provide electrical communication. A sealant or adhesive may be used to isolate and protect the connections of the conductors 154 and the contacts 156.

The sensor housing 112 may then be introduced over the distal end of the hypotube 110 to cover the sensor mount 134 and to increase the rigidity of the hypotube 110 in the region of the sensor mount 134. The sensor housing 112 may be aligned so that its window overlies the sensor 150 and the distal and proximal ends lie upon the fully cylindrical portions at the distal and proximal sides of the sensor mount 134. The sensor housing 112 may be then secured to the hypotube using an adhesive or weld or other method.

With the sensor housing 112 and the flex wire 172 in place on the hypotube 110, the coil 170 and the distal cap 174 may then be introduced to the hypotube 110. The distal cap 174 may be formed or soldered in place over the distal end of the coil 170 to form a rounded end. In embodiments where the distal cap 174 is a separate component, the distal cap may be secured using an adhesive, a weld, or other attachment method. In some aspects, the distal cap 174 is screwed or threaded onto the coil 170. With the distal cap on the coil 170, the coil may be introduced over the flex wire 172 and secured to the hypotube 110. As discussed above, the coil may be secured by an adhesive, may be welded, soldered, or otherwise bonded to the hypotube 110. In some embodiments, the coil is threaded onto the hypotube.

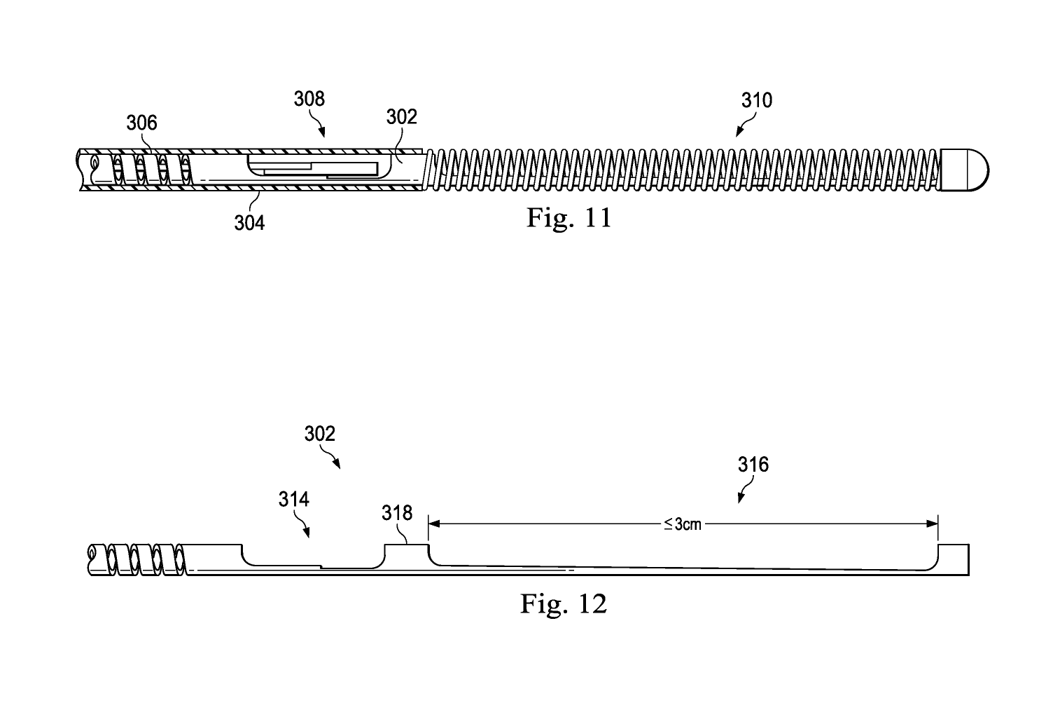

FIGS. 11-15 show another embodiment of a distal end of a guidewire that may be used as a part of the system 10 discussed above. FIG. 11 shows a distal region of a guidewire referenced herein by the numeral 300. The guidewire 300 includes a hypotube 302, a sensor housing 304, a polymer sleeve 306, a sensor assembly 308, and a distal tip 310. Much of the description above applies to the elements in the guidewire 300 and that description will not be repeated here.

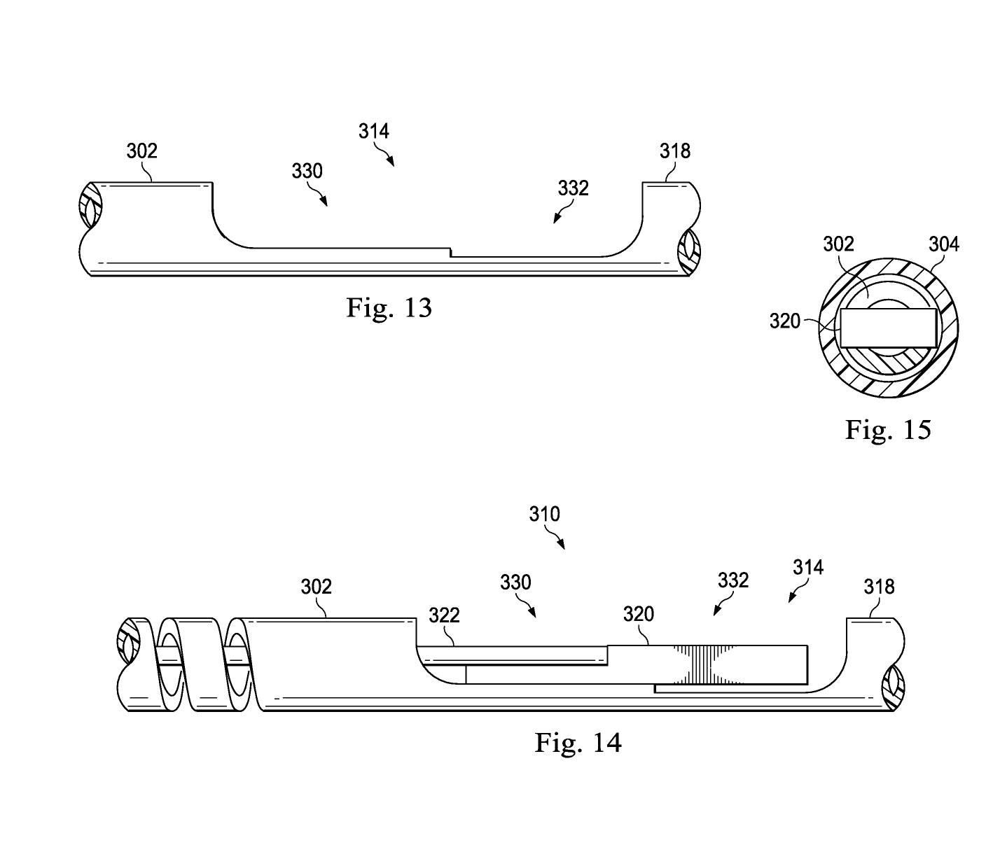

Referring to FIGS. 12 and 13, the hypotube 302 includes an integrated sensor mount 314 and an integrated flex wire 316. In this embodiment, the hypotube 302 also includes spiral cuts increasing the flexibility of the hypotube 302 proximal of the sensor mount 314. The integrated flex wire 316 extends from a fully-cylindrical portion 318 disposed between the integrated sensor mount 314 and the flex wire 316 and forms a part of the distal tip 310.

With reference to FIG. 14, the sensor assembly 308 includes a sensor carried on a sensor block 320 and conductors 322. The sensor block 320 carries the sensor in the manner discussed above.

FIG. 13 shows the integrated sensor mount 314 in greater detail, and FIG. 14 shows the sensor assembly 308 disposed in the sensor mount 314. The sensor mount 314 in FIG. 13 includes a first region 330 and a second region 332 having different heights, with at least a portion of the sensor assembly 308 arranged to be disposed on the first level of the first region 330 and extend axially as a cantilever over the second level of the second region 332. The distal tip 310 shown in FIG. 11 includes the flex wire 316, a coil 340, and a distal cap 342.

FIG. 15 shows an end view of the sensor block 320 disposed on the sensor mount 314. As can be seen, the hypotube sensor mount 314 has a width across the hypotube lumen and the sensor block 320 of the sensor assembly 308 has a width greater than the width of the sensor mount 314. Accordingly, the sensor block 320 lies on the walls of the sensor mount 314 in the manner discussed above. In this embodiment, the sensor housing 304 is a thin-walled sensor housing. Accordingly, it does not directly engage against the sensor block 320, and is carried on the cylindrical portions of the hypotube 302.

Using the integrated sensor mounts disclosed herein may increase the repeatability and consistency of sensor placement during the manufacturing process. This may provide a more consistent product to the surgeons increasing surgeon satisfaction and simplifying the assembly process.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

I claim:

1. A guidewire system for treating a patient, comprising:
a sensor assembly for detecting a physiological characteristic of a patient;
a hypotube having an integrated sensor mount formed therein for predictably locating the sensor during assembly, the sensor mount having a first mechanical stop configured to limit movement of the sensor in at least a first dimension and a second mechanical stop configured to limit movement of the sensor in at least a second dimension; and
a sensor housing disposed about the sensor mount and configured to reinforce the hypotube at the sensor mount.

2. The guidewire system of claim 1, wherein the sensor assembly has a width greater than a width of a lumen of the hypotube and the sensor mount comprises walls of the hypotube such that a portion of the sensor assembly lies directly on the walls of the hypotube.

3. The guidewire system of claim 1, wherein the first mechanical stop is configured to maintain the sensor at a desired height, and wherein the second mechanical stop is configured to maintain the sensor at a desired axial location.

4. The guidewire system of claim 1, wherein the sensor housing comprises a window configured to provide fluid communication between the sensor and an environment outside the sensor housing.

5. The guidewire system of claim 1, wherein the integrated sensor mount comprises a cutout having a first level and a second level, the sensor assembly being disposed on the first level, the second level being lower than the first level.

6. The guidewire system of claim 5, wherein the sensor assembly extends longitudinally from the first level to a cantilevered position over the second level.

7. The guidewire system of claim 5, wherein the integrated sensor mount comprises a third level, the first and third level forming the first mechanical stop.

8. The guidewire system of claim 1, wherein the second mechanical stop is formed of upwardly facing surfaces of walls of the hypotube.

9. The guidewire system of claim 1, wherein the hypotube is formed of Nitinol.

10. The guidewire system of claim 9, wherein the sensor housing is formed of stainless steel.

11. The guidewire system of claim 1, comprising a flexible member introduced into the sensor mount, the flexible member comprising a more flexible distal end a less flexible proximal end, the flexible member extending from a distal end of the hypotube.

12. The guidewire system of claim 11, wherein the flexible member comprises an anchoring element disposed at a proximal end, the anchor member preventing the proximal end from passing out of the distal end of the hypotube.

13. A guidewire system for treating a patient, comprising:
a sensor assembly for detecting a physiological characteristic of a patient, the sensor assembly having a portion having a first width;
a hypotube having an integrated sensor mount formed therein for predictably locating the sensor during assembly, the hypotube having a lumen and the sensor mount being formed of opposing walls of the hypotube, the distance between the opposing walls being a second width, the first width of the sensor assembly being greater than the second width between the opposing walls of the hypotube such that a portion of the sensor assembly lies directly on the walls of the hypotube; and
a sensor housing disposed about the sensor mount and configured to reinforce the hypotube at the sensor mount.

14. The guidewire system of claim 13, wherein the sensor mount comprises a first mechanical stop configured to limit movement of the sensor in at least a first dimension and a second mechanical stop configured to limit movement of the sensor in at least a second dimension.

15. The guidewire system of claim 13, wherein the sensor housing comprises a window configured to provide fluid communication between the sensor and an environment outside the sensor housing.

16. The guidewire system of claim 13, wherein the integrated sensor mount comprises a cutout having a first level and a second level, the sensor assembly lying on walls of the hypotube forming the first level, the second level being lower than the first level.

17. The guidewire system of claim 16, wherein the sensor assembly extends longitudinally from the first level to a cantilevered position over the second level.

18. The guidewire system of claim 17, wherein the integrated sensor mount comprises a third level, the first and third level forming a mechanical stop.

19. A method of building a guidewire comprising:
providing a sensor mount in a hypotube sized for introduction to a patient's vasculature when treating a medical condition;
placing a sensor assembly on the sensor mount in the hypotube, the sensor mount having a surface configured to cooperate with the sensor assembly to locate the sensor assembly at a desired height by limiting movement of the sensor assembly in a first direction;
orienting the sensor assembly to abut a mechanical stop that limits movement of the sensor assembly in a second dimension;
securing the sensor in place; and
introducing the sensor mount into a sensor housing having a window formed therein to increase the rigidity of the hypotube at the sensor mount.

20. The method of claim 19, comprising aligning the sensor assembly with edges of the sensor mount to orient the sensor assembly in a third dimension.

21. The method of claim 19, comprising introducing a flex wire through an end of the hypotube to provide a flexible distal tip of the guidewire.

22. A method of building a guidewire comprising:
providing a sensor mount in a hypotube sized for introduction to a patient's vasculature when treating a medical condition;
placing a sensor assembly on the sensor mount in the hypotube, the sensor assembly having a portion having a first width and spanning a lumen in the hypotube such that a portion of the sensor assembly lies directly on opposing walls of the hypotube; and
securing the sensor in place; and
introducing the sensor mount into a sensor housing having a window formed therein to increase the rigidity of the hypotube at the sensor mount.

23. The method of claim 22, comprising aligning the sensor assembly with edges of the sensor mount to orient the sensor assembly in a third dimension.

24. The method of claim 22, wherein the integrated sensor mount comprises a cutout having a first level and a second level, and wherein introducing the sensor assembly includes placing the sensor assembly so that it extends longitudinally from the first level to a cantilevered position over the second level.

25. A guidewire system for treating a patient, comprising:
a pressure sensor for detecting a physiological characteristic of a patient;
a hypotube having an integrated sensor mount formed therein for predictably locating the pressure sensor during assembly, the sensor mount being disposed between two fully cylindrical portions of the hypotube, the sensor mount having a first mechanical stop configured to limit movement of the sensor in at least a first dimension and a second mechanical stop configured to limit movement of the sensor in at least a second dimension, wherein the first mechanical stop is configured to maintain the sensor at a desired height, and wherein the second mechanical stop is configured to maintain the sensor at a desired axial location, the sensor having an axial length greater than an axial length of the first mechanical stop so that the sensor extends as a cantilever from the first mechanical stop; and
a sensor housing disposed about the sensor mount and configured to reinforce the hypotube at the sensor mount, the sensor housing comprising a window configured to provide fluid communication between the sensor and an environment outside the sensor housing;
conductors extending inside the hypotube from the sensor to the proximal end of the hypotube; and
a stiffening portion distally extending from one of the two fully cylindrical portions of the hypotube.

26. The guidewire system of claim 25, wherein the first mechanical stop is an upper surface of walls of the hypotube.
